(19) United States
(12) Patent Application Publication
(10) Pub. No.: US 2006/0067991 A1
(43) Pub. Date: Mar. 30, 2006

(54) Non-absorbent articles for inhibiting the production of exoproteins

(75) Inventors: Rae Ellen Syverson, Fond du Lac, WI (US); Kristin A. Goerg-Wood, Sherwood, WI (US); Daniel J. Devine, Menasha, WI (US)

Correspondence Address: SENNIGER POWERS, ONE METROPOLITAN SQUARE, 16TH FLOOR, ST LOUIS, MO, 63102, US

(73) Assignee: Kimberly-Clark Worldwide, Inc.

(21) Appl. No.: 10/955,858

(22) Filed: Sep. 30, 2004

(51) Int. Cl. A61K 9/70

(52) U.S. Cl. 424/443

(57) ABSTRACT

Non-absorbent articles for inhibiting the production of exoproteins from Gram positive bacteria are disclosed. The non-absorbent articles include an effective amount of a precursor compound having the general formula: 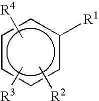
wherein R¹ is selected from the group consisting of 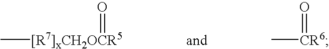
R⁷ is —OCH₂—; X is 0 or 1; R⁵ is a substituted or unsubstituted aromatic ring or a monovalent saturated or unsaturated, substituted or unsubstituted, branched or straight chain hydrocarbyl moiety that may or may not be substituted with hetero atoms; R⁶ is selected from the group consisting of an amino acid, a methyl ester of an amino acid, and an ethyl ester of an amino acid; R², R³, and R⁴ are independently selected from the group consisting of H, OH, COOH.

### BACKGROUND OF INVENTION

The present invention relates to the inhibition of exoprotein production in and around a woman's vagina in association with a non-absorbent article. More particularly, the present invention relates to the incorporation of a precursor compound into or onto a non-absorbent article such that upon use, the precursor compound can be hydrolyzed by enzymatic activity in and around the vagina to produce an active species that reduces the production of exoproteins from bacteria.

There exists in the female body a complex process that maintains the vagina and physiologically related areas in a healthy state. In a female between the age of menarche and menopause, the normal vagina provides an ecosystem for a variety of microorganisms. Bacteria are the predominant type of microorganism present in the vagina, and most women harbor about 10⁹ bacteria per gram of vaginal fluid. The bacterial flora of the vagina is comprised of both aerobic and anaerobic bacteria. The more commonly isolated bacteria are Lactobacillus species, Corynebacteria species, Gardnerella vaginalis, Staphylococcus species, Peptococcus species, aerobic and anaerobic Streptococcus species, and Bacteroides species. Other microorganisms that have been isolated from the vagina on occasion include yeast (Candida albicans), protozoa (Trichomonas vaginalis), mycoplasma (Mycoplasma hominis), chlamydia (Chlamydia trachomatis), and viruses (Herpes simplex). These latter organisms are generally associated with vaginitis or venereal disease, although they may be present in low numbers without causing symptoms.

Physiological, social, and idiosyncratic factors affect the quantity and species of bacteria present in the vagina. Physiological factors include age, day of the menstrual cycle, and pregnancy. For example, microorganisms present in the vagina throughout the menstrual cycle can include lactobacilli, corynebacterium, ureaplasma, and mycoplasma. The number of microoganisms and the types of microorganisms are unique to an individual. Social and idiosyncratic factors include method of birth control, sexual practices, systemic disease (e.g., diabetes), and medications.

Bacterial proteins and metabolic products produced in the vagina can affect other microorganisms and the human host. For example, the vagina between menstrual periods is mildly acidic having a pH ranging from about 3.8 to about 4.5. This pH range is generally considered the most favorable condition for the maintenance of normal flora. At that pH, the vagina normally harbors the numerous species of microorganisms in a balanced ecology. These microorganisms play a beneficial role in providing protection and resistance to infection and make the vagina inhospitable to some species of bacteria such as Staphylococcus aureus (S. aureus). The low pH is a consequence of the growth of lactobacilli and their production of acidic products. Microorganisms in the vagina can also produce antimicrobial compounds such as hydrogen peroxide and bactericides directed at other bacterial species. One example is the lactocins, bacteriocin-like products of lactobacilli directed against other species of lactobacilli.

Some microbial products produced in the vagina may negatively affect the human host. For example, S. aureus can produce and excrete into its environment a variety of exoproteins including enterotoxins, Toxic Shock Syndrome Toxin-1 (TSST-1), and enzymes such as esterase and amidase. When absorbed into the bloodstream of the host, TSST-1 may lead to the development of Toxic Shock Syndrome (TSS) in non-immune humans.

S. aureus is found in the vagina of approximately 16% of healthy women of menstrual age. Not all strains of S. aureus can produce TSST-1. Approximately 25% of these women will harbor TSST-1 producing S. aureus. TSST-1 and some of the Staphylococcal enterotoxins have been identified as causing TSS in humans.

Symptoms of TSS generally include fever, diarrhea, vomiting and a rash followed by a rapid drop in blood pressure. Multiple organ failure occurs in approximately 6% of those who develop the disease. S. aureus does not initiate TSS as a result of the invasion of the microorganism into the vaginal cavity. As S. aureus grows and multiplies, it can produce TSST-1. Only after entering the bloodstream does TSST-1 act systemically and produce the symptoms attributed to TSS.

Menstrual fluid has a pH of about 7.3. During menses, the pH of the vagina moves toward neutral and can become slightly alkaline. This change permits microorganisms whose growth is inhibited by an acidic environment to proliferate. For example, S. aureus is more frequently isolated from vaginal swabs during menstruation than from swabs collected between menstrual periods.

When S. aureus is present in an area of the human body that harbors a normal microbial population such as the vagina, it may be difficult to eradicate the S. aureus bacterium without harming members of the normal microbial flora required for a healthy ecosystem. Typically, antibiotics that kill S. aureus are not an option for use in products inserted into the vagina because of their effect on the normal vaginal microbial flora. An alternative to complete eradication is technology designed to prevent or substantially reduce the bacterium's ability to produce toxins.

There have been numerous attempts to reduce or eliminate pathogenic microorganisms and menstrually occurring TSS by incorporating one or more biostatic, biocidal, and/or detoxifying compounds into vaginal products. For example, L-ascorbic acid has been applied to a menstrual tampon to detoxify toxin found in the vagina. Others have incorporated monoesters and diesters of polyhydric aliphatic alcohols, such as glycerol monolaurate, as biocidal compounds (see, e.g., U.S. Pat. No. 5,679,369). Still others have introduced other non-ionic surfactants, such as alkyl ethers, alkyl amines, and alkyl amides as detoxifying compounds (see, e.g., U.S. Pat. Nos. 5,685,872, 5,618,554, and 5,612,045).

One significant problem associated with some of the above previous attempts is that the compounds used may be highly volatile during incorporation into non-absorbent articles and during further manufacturing processes. Specifically, it has been discovered that compounds, such as some aromatics, terpenes, and isoprenoids, are volatilized completely out of a non-absorbent product during high temperature manufacturing steps. Also, some compounds may have volatility issues during storage prior to use by the consumer.

As such, there continues to be a need for non-absorbent products comprising inhibitory compounds that will effectively inhibit the production of exoproteins, such as TSST-1, from Gram positive bacteria without being substantially harmful to the natural flora found in the vaginal area. Additionally, these inhibitory compounds need to maintain activity even in the presence of enzymes such as lipase, esterase, and amidase, which can have adverse effects on potency and which may also be present in the vagina. It is desirable that the compounds have low volatility and remain in the product throughout manufacturing, storage, and transportation in order to deliver an effective inhibitor to the consumer.

### SUMMARY OF THE INVENTION

The present invention is directed to a non-absorbent product that inhibits the production of exoprotein from Gram positive bacteria. More specifically, the present invention is directed to a non-absorbent substrate, such as a tampon applicator, incorporating one or more precursor compounds that are formed by linking one or more aromatic compounds to one or more secondary compounds via an ester or amide bond. Once introduced into the vagina, these precursor compounds can be hydrolyzed by enzymes produced by the natural vaginal flora resulting in an active species that can inhibit the production of exoproteins from Gram positive bacteria without substantially affecting the flora present in the vagina. In some embodiments, the precursor compound itself may also inhibit the production of exoproteins from Gram positive bacteria.

Therefore, the present invention is directed to an exoprotein inhibitor for inhibiting the production of exoproteins from Gram positive bacteria. The exoprotein inhibitor comprises a non-absorbent substrate suitable for insertion into the vagina and having deposited thereon an effective amount of a precursor compound having the general formula: 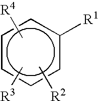
wherein R¹ is 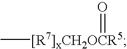
R⁷ is —OCH₂—; X is 0 or 1; R⁵ is a substituted or unsubstituted aromatic ring or a monovalent saturated or unsaturated, substituted or unsubstituted, branched or straight chain hydrocarbyl moiety that may or may not be substituted with heteroatoms; R², R³, and R⁴ are independently selected from the group consisting of H, OH, COOH, wherein upon hydrolysis the precursor compound is capable of producing an active species effective in inhibiting the production of exoprotein from Gram positive bacteria.

The present invention is further directed to an exoprotein inhibitor for inhibiting the production of exoproteins from Gram positive bacteria. The exoprotein inhibitor comprises a non-absorbent substrate suitable for insertion into the vagina and having deposited thereon an effective amount of a precursor compound having the general formula: 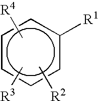
wherein R¹ is 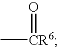
R⁶ is selected from the group consisting of an amino acid, a methyl ester of an amino acid, and an ethyl ester of an amino acid; R², R³, and R⁴ are independently selected from the group consisting of H, OH, COOH, wherein upon hydrolysis the precursor compound is capable of producing an active species effective in inhibiting the production of exoprotein from Gram positive bacteria.

The present invention is further directed to a tampon applicator for inhibiting the production of exoprotein from Gram positive bacteria. The tampon applicator comprises a non-absorbent material suitable for insertion into a vagina and having deposited thereon an effective amount of a precursor compound having the general formula: 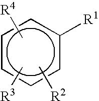
wherein R¹ is 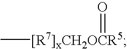
R⁷ is —OCH₂—; X is 0 or 1; R⁵ is a substituted or unsubstituted aromatic ring or a monovalent saturated or unsaturated, substituted or unsubstituted, branched or straight chain hydrocarbyl moiety that may or may not be substituted with heteroatoms; R², R³, and R⁴ are independently selected from the group consisting of H, OH, COOH, wherein upon hydrolysis the precursor compound is capable of producing an active species effective in inhibiting the production of exoprotein from Gram positive bacteria.

The present invention is further directed to a tampon applicator for inhibiting the production of exoprotein from Gram positive bacteria. The tampon applicator comprises a non-absorbent material suitable for insertion into a vagina and having deposited thereon an effective amount of a precursor compound having the general formula: 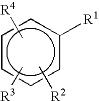
wherein R¹ is 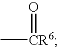
R⁶ is selected from the group consisting of an amino acid, a methyl ester of an amino acid, and an ethyl ester of an amino acid; R², R³, and R⁴ are independently selected from the group consisting of H, OH, COOH, wherein upon hydrolysis the precursor compound is capable of producing an active species effective in inhibiting the production of exoprotein from Gram positive bacteria.

The present invention is further directed to a douche formulation for inhibiting the production of exoprotein from Gram positive bacteria located in and around a woman's vagina. The douche formulation comprises a vaginal cleansing formulation comprising a pharmaceutically acceptable carrier and an effective amount of a precursor compound having the general formula: 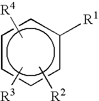
wherein R¹ is 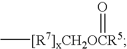
R⁷ is —OCH₂—; X is 0 or 1; R⁵ is a substituted or unsubstituted aromatic ring or a monovalent saturated or unsaturated, substituted or unsubstituted, branched or straight chain hydrocarbyl moiety that may or may not be substituted with heteroatoms; R², R³, and R⁴ are independently selected from the group consisting of H, OH, COOH, wherein upon hydrolysis the precursor compound is capable of producing an active species effective in inhibiting the production of exoprotein from Gram positive bacteria, and wherein the vaginal cleansing formulation is suitable for use in a woman's vagina.

The present invention is further directed to a douche formulation for inhibiting the production of exoprotein from Gram positive bacteria located in and around a woman's vagina. The douche formulation comprises a vaginal cleansing formulation comprising a pharmaceutically acceptable carrier and an effective amount of a precursor compound having the general formula: 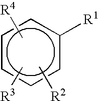
wherein R¹ is 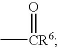
R⁶ is selected from the group consisting of an amino acid, a methyl ester of an amino acid, and an ethyl ester of an amino acid; R², R³, and R⁴ are independently selected from the group consisting of H, OH, COOH, wherein upon hydrolysis the precursor compound is capable of producing an active species effective in inhibiting the production of exoprotein from Gram positive bacteria, and wherein the vaginal cleansing formulation is suitable for use in a woman's vagina.

Other features and advantages of this invention will be in part apparent and in part pointed out hereinafter.

### DETAILED DESCRIPTION OF THE PREFERRED EMBODIMENTS

The present invention is generally directed to non-absorbent articles comprising a precursor compound that upon hydrolysis produces an active species capable of inhibiting the production of exoproteins from Gram positive bacteria. Specifically, the present invention relates to a non-absorbent product comprising a precursor compound formed by linking an aromatic compound to a second compound by an ester or amide bond that can be readily hydrolyzed by enzymatic action once inside of the vagina to produce an active species and a second compound. The active species have been found to substantially inhibit the production of exoproteins, such as TSST-1, from Gram positive bacteria. Typically, the precursor compound itself may also inhibit the production of exoproteins from Gram positive bacteria. Additionally, the precursor compounds can be used in combination with surface-active agents such as, for example, myreth-3 myristate, glycerol monolaurate, and/or laureth-4, to substantially inhibit the production of exoproteins from Gram positive bacteria.

This invention will be described herein in detail in connection with a tampon applicator, but will be recognized by one skilled in the art to be applicable to other non-absorbent articles, devices, and/or products, such as non-absorbent incontinent devices, barrier birth control devices, non-absorbent contraceptive devices, and douches.

As used herein, the phrase “non-absorbent article” generally refers to substrates or devices which include an outer layer formed from a substantially hydrophobic material which repels fluids, such as menses, blood products, and the like. Suitable materials for constructing the non-absorbent articles of the present invention include, for example, rubber, plastic, and cardboard.

Typically, a tampon applicator includes an outer tube, which is preferably in the form of a hollow tube. The tube is formed from paper, paperboard, cardboard, plastic, thermoplastic film, aqueous coating or a combination thereof. If paper, paperboard or cardboard is used, it can be coated with a wax or water-insoluble polymer to render it water-resistant. Suitable plastic materials include polyolefins, such as low density polyethylene and low density polypropylene. The outer tube should have sufficient strength and rigidity to prevent collapse under normal vaginal pressures. The outer tube can also be formed into a cylindrical shape having a longitudinal seam or be spirally or convolutely wound. The outer tube has a relatively small diameter of about 10 mm to about 20 mm.

The outer tube has first and second spaced apart ends. The outer tube is formed from at least two distinct layers, which may be constructed of equal or different board weight. The layers can be made from different materials, for example, paperboard and film, or be made from similar material having different properties, for example, different board weight. In one embodiment, the exterior layer can be formed from a thin coated paperboard of about 0.06 mm or from a film material having a thickness of about 0.01 mm while one or more inner layers can be formed from a non-coated material having a higher board weight. The exterior layer can consist of a high gloss, coated paper, which is water-degradable or water-dispersible. Alternatively, the exterior layer could have different finishes, such as semi-gloss or a satin finish. The coating on the outer tube can be selected from a wide variety of materials. Suitable coatings may include polyethylene, polypropylene, polyvinylidene chloride and polychloride alcohol. The exterior layer can also be lubricated or contain an additive. Suitable lubricants and additives include any of the pharmaceutically accepted lubricants or additives conventionally used in tampon applicators. Such lubricants and additives include organic compounds, long chain aliphatic groups, such as derivatives of fatty acids, for example, stearamides and oleamides.

Paper used in the construction of the tampon applicator should have a board weight per layer of from between about 20 pounds to about 200 pounds per ream, suitably, between about 25 pounds to about 100 pounds per ream, and more suitably, from about 30 pounds to about 50 pounds per ream. A “ream” is defined as material having dimensions of 24 inches (609.6 mm) by 36 inches (914.4 mm) by 500 sheets. Each paperboard layer should have a thickness of less than about 0.4 mm, suitably, from about 0.04 mm to about 0.2 mm, and more suitably, from about 0.05 mm to about 0.16 mm. Typically, the exterior layer will be thinner than the interior paperboard layer(s).

If one of the layers is made from a thermoplastic film, it can be polyethylene. A suitable polyethylene film has high slip characteristics and a low density. The thermoplastic film should be thin, less than about 0.1 mm, suitably about 0.010 mm to about 0.050 mm, and more suitably about 0.012 mm to about 0.040 mm. Other kinds of films can also be used. Such films include cellulose ether selected from the group of aliphatic and aromatic ethers; films having ethylcellulose as the essential base constituent, or films of methyl cellulose; flexible, highly plasticized cellulose acetate, formate and similar other alkyl esters; vinyl vinylidene chloride or rubber hydrochloride, as for example, Pliofilm™., or vinylite resin.

The thermoplastic film can be clear or opaque. The film may run the entire length of the outer tube or only extend along a portion thereof. The film can be on the exterior surface of the outer tube or be one of the inner layers.

The layers of the outer tube can be held together by an adhesive, such as glue, or by heat, pressure, ultrasonics, etc. The adhesive can be either water-soluble or water-insoluble. A water-soluble adhesive is preferred for environmental reasons in that the outer tube will quickly break apart when it is immersed in water. Such immersion will occur should the outer tube be disposed of by flushing it down a toilet.

The outer tube is sized and configured to house an absorbent catamenial tampon. The inside diameter of the outer tube is sized to accommodate typical size tampons. Usually, the inside diameter of the outer tube is less than about 0.75 inches (about 19 mm) and more suitably, less than about 0.625 inches (about 16 mm). Although the exterior diameter of tampons does vary, most tampons utilized by women have an external diameter of less than about 0.75 inches (about 19 mm).

The outer tube should have a substantially smooth exterior surface, which will facilitate insertion of the tampon applicator into a woman's vagina. When the surface of the exterior layer is smooth and/or slippery, the outer tube will easily slide into a woman's vagina without subjecting the internal tissues of the woman's vagina to abrasion. The outer tube can be coated to give it a high slip characteristic. Wax, polyethylene, a combination of wax and polyethylene, cellophane and clay are representative coatings that can be applied to the exterior layer to facilitate comfortable insertion. The outer tube can be a straight, elongated cylindrical tube formed on a central longitudinal axis. It is also possible to form the outer tube into an arcuate shape. The arcuate or curved shape can assist in providing comfort when inserting the outer tube into a woman's vagina. With a curved tampon applicator, it is possible to employ a curved tampon which again may be more comfortable for some women to use since the shape of the tampon may better fit the curvature of a woman's vagina.

Integrally formed on the first end of the outer tube and extending outwardly therefrom is an insertion tip. The insertion tip is designed to facilitate insertion of the outer tube into a woman's vagina in a comfortable manner. The insertion tip should be made of a thin, flexible material or membrane, which resists rapid absorption of vaginal fluid during the period of insertion of the tampon applicator into the woman's vagina. The insertion tip can be constructed of paper, paperboard or film material. When the outer tube has only two layers, the insertion tip should be formed out of the layer having the lower board weight. The lower board weight layer is normally the thinner layer. A film material is preferred because it is thin, soft and flexible. Suitable materials for the insertion tip include a thin bonded nonwoven fabric layer coated with low density polyethylene, plasticized polyvinyl chloride or polyurethane. The insertion tip can also contain a coating or impregnation, which inhibits any substantial absorption of vaginal fluids. The coating may be an oil, a wax, or an acceptable organic compound. Alternatively, the insertion tip can be self-lubricating. Such materials can be made of a polymer that inherently provides the outer surface with a low coefficient of friction. Typical polymers of this type are fluorinated, such as polytetrafluoroethylene (PTFE), fluorinated ethylene-propylene (FEP) and polyethyleneoxide (PEO).

The insertion tip should have an outside diameter, which is approximately equal to or less than the outside diameter of the outer tube. It should be noticed that when the diameter is less than that of the outer tube, the difference should be small so that the end of the exterior layer cannot be felt by the woman during insertion. Generally, the insertion tip has a diameter that is less than the diameter of the outer tube. The insertion tip can be configured to be rounded, semi-spherical or frusto-conical. Other nose or dome-like shapes can also be utilized. The rounded configuration of the insertion tip functions to prevent the forward end of the tampon from exerting an abrasive action upon the wall of the vagina as would be the case if it was uncovered.

The insertion tip is formed from at least one of the layers, which form the outer tube and can be formed from more than one layer if desired, provided it has less thickness. The insertion tip can be formed from at least one less layer than the number of layers from which the outer tube is constructed. The insertion tip has a thickness that is less than the thickness of the outer tube so as to assure that it is soft and flexible. The thickness of the insertion tip should be less than about 50% of the thickness of the outer tube, suitably less than about 75% of the thickness of the outer tube, and more suitably, less than about 80% of the thickness of the outer tube.

The insertion tip may have a plurality of soft and flexible petals, which are arranged to form a dome-shaped nose. The petals are separated by narrow slots. The petals are capable of radially flexing or bending outward to provide an enlarged opening through which the tampon can exit when it is pushed forward by the inner tube. Either an even or an odd number of petals can be used but preferably, there are an odd number of petals, such as 3, 5, 7, etc. because an odd number of petals will prevent the outer tube from collapsing or flattening after the tampon has been expelled. By preventing the outer tube from collapsing, one can be assured that the vaginal tissue will not be pinched when the tampon applicator is removed from the user's vagina. For example, the insertion tip will contain five petals, each having an elongated, approximately truncated shape with a rounded end and each being about 7/16 of an inch (about 11.1 mm) in length.

As stated above, the tampon applicator includes an inner tube. The inner tube, like the outer tube, can be a spirally wound, convolutely wound or longitudinally seamed, hollow tube constructed from paper, paperboard, cardboard, plastic, film, aqueous coating or a combination thereof. The inner tube can also be formed into a hollow tube by overlapping the material upon itself. The inner tube can be constructed of the same material as the outer tube or it can be made out of a different material. Furthermore, the inner tube could be constructed as a laminate having two or more plies which are then spirally wound, convolutely wound or longitudinally seamed into a cylindrical tube. Either a wound tube or a longitudinally seamed tube is preferred because the finished tube will have a wall with a constant thickness. However, some manufacturers may prefer to construct the inner tube as a solid stick or use some other unique shape. The inner tube also has a distal or free end onto which the user's forefinger can rest for facilitating movement of the inner tube into the outer tube. The distal end thereby functions as a seat for the forefinger. It is also possible to form an enlarged ring or flange on the distal end of the inner tube to provide for a larger contact surface.

The inner tube functions by telescopically moving into the outer tube. As the inner tube is pushed into the outer tube, the tampon is forced forward against the insertion tip. The contact by the tampon causes the petals to radially open to a diameter, which is sufficient to allow the tampon to be expelled from the outer tube. With the tampon properly positioned in the woman's vagina, the tampon applicator is withdrawn and discarded.

The weight of a tampon applicator will depend on the size and absorbency of the tampon. For example, a longer, more absorbent tampon will be heavier than a shorter, less absorbent tampon. Typically, an applicator suitable for use in the present invention with a regular absorbency tampon will weigh about 3.62 grams; an applicator suitable for use with a super absorbency or super plus absorbency tampon will weigh about 4.12 grams.

Other suitable non-absorbent articles for the present invention include, for example, non-absorbent incontinent devices, non-absorbent contraceptive devices, such as barrier birth control devices, and douches. As used herein, “non-absorbent incontinent device” refers to a device designed to be inserted into a woman's vagina and expanded so as to relieve or eliminate the involuntary passage of urine through the uretha from the bladder. The expansion of the non-absorbent urinary incontinent device provides a stable backdrop to the musculature and body tissue located near the urethro-vaginal myofascial area and causes the urethra to be compressed upon itself during episodes of increased intra-abdominal pressure. In addition, the expansion of the incontinent device in the vagina will assist the urinary sphincter muscle in maintaining a circular cross-sectional configuration. One example of a suitable non-absorbent incontinent device is disclosed in U.S. Pat. No. 6,679,831 issued to Zunker, et al. (Jan. 20, 2004).

Suitable non-absorbent contraceptive devices, such as barrier birth control devices, for the present invention are known in the art. For example, U.S. Pat. No. 4,711,235 issued to Willis (Dec. 8, 1987) discloses a device comprising a ring having a central sheet of resilient impermeable material sandwiched between two layers of foam rubber. The foam rubber traps sperm and bacteria in a vaginal recess for a time sufficient to be destroyed by the normal acid pH of the vaginal secretions. The ring defines the sheet material into a cup-shape with four sides, one pair of opposing sides including a wire core inwardly bent and another pair of sides with one outwardly rounded and the other having a fork configuration. The resilient impermeable material is typically a neoprene non-absorbent rubber material.

As stated above, the non-absorbent articles of the present invention comprise an effective amount of a precursor compound that, upon hydrolysis, produces an active species that can substantially inhibit the production of exoprotein by Gram positive bacterium and, specifically, the production of TSST-1 from S. aureus bacterium. As used herein, the term “precursor compound” means a compound that is introduced into and/or onto a non-absorbent article that is capable of undergoing hydrolysis inside and/or adjacent to the vagina to produce an active species capable of inhibiting the production of exoproteins from Gram positive bacteria. The precursor compounds are formed by linking an aromatic compound to a second compound with an ester or amide bond. The ester or amide bond contained in the precursor compound is hydrolyzed by enzymes, such as lipase, esterase, and/or amidase, which are produced by bacteria found in the natural vaginal flora, resulting in an active species that can inhibit exoprotein production from Gram positive bacteria. Along with the active species produced, the hydrolysis reaction produces a second compound that is not critical to the function of the active species. In some embodiments, the second compound will be identical or similar to compounds naturally occurring in the human body. Although, as noted above, the second compound is not critical, the precursor compound should be designed such that upon hydrolysis the formed second compound is not substantially harmful to the vagina or the bacteria located therein.

During the hydrolysis process, the precursor compound is slowly broken down into the active species and the secondary compound; and, as noted above, both the precursor compound and the active species can inhibit the production of exoproteins from Gram positive bacteria. This property is advantageous as it allows for long-lasting continuous inhibition of exoprotein production by Gram positive bacteria. The precursor compounds of the present invention can inhibit the production of exoproteins prior to hydrolysis; and then, as the precursor compounds are hydrolyzed, the active species are produced, which can further inhibit exoprotein production.

The precursor compounds described herein and suitable for introduction into and/or onto a non-absorbent article are both substantially stable in and/or on the non-absorbent article both throughout the manufacturing processes and during shelf storage. Stated another way, the precursor compounds are not easily volatilized off of or out of the non-absorbent article during high temperature manufacturing processes or during shipment and storage. This property of the precursor compounds is highly desirable as it is important for the precursor compound to remain in or on the non-absorbent article product in an effective amount for ultimate use by the consumer. As noted above, volatility of active compounds from non-absorbent articles have been problematic in the past and can result in a non-absorbent product devoid of active compound.

Without being bound to a particular theory, it is believed that the precursor compounds of the present invention may remain in or on the non-absorbent articles in an increased amount as compared to prior ingredients due to their increased molecular weight. Although the precursor compounds of the present invention have a generally higher molecular weight as compared to some active ingredients utilized in the past, they are suitably hydrolyzed in the body to produce highly desirable active species, such as benzyl alcohol and benzoic acid, which are highly effective in inhibiting the production of exoprotein by Gram positive bacteria.

In addition to having a reduced volatility and being capable of remaining in and/or on a non-absorbent product throughout manufacturing, shipping, and storage, the precursor compounds described herein, along with the hydrolyzed active species and second compounds produced in the body, do not kill a substantial amount of naturally occurring bacteria found in the vagina. This property is significant as the complete, or substantially complete, non-specific killing of bacteria located in and around the vagina can be very harmful for the host as natural flora are required to maintain a healthy vagina. In contrast to agents such as antibacterials or antivirals that non-specifically kill all microorganisms in the vagina, the precursor compounds and hydrolyzed compounds produced in and around the vaginal cavity do not have a substantial killing effect on bacteria at the concentration incorporated into the product; but the active species produced by hydrolysis can substantially inhibit the production of exotoxins from Gram positive bacteria.

In one embodiment of the present invention, the precursor compounds have the general chemical structure: 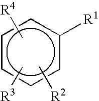
wherein R¹ is 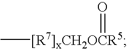
R⁷ is —OCH₂—; X is 0 or 1; R⁵ is a substituted or unsubstituted aromatic ring or a monovalent saturated or unsaturated, substituted or unsubstituted, branched or straight chain hydrocarbyl moiety that may or may not be substituted with heteroatoms; and R², R³, and R⁴ are independently selected from the group consisting of H, OH, COOH.

As noted above, the hydrocarbyl moieties described herein include both straight chain and branched chain hydrocarbyl moieties that may or may not be substituted with various substituents such as, for example, hydroxyl groups. Additionally, the hydrocarbyl moiety may or may not be interrupted with hetero atoms. Hetero atoms that can interrupt the hydrocarbyl moiety include, for example, oxygen, nitrogen, and sulfur. In one embodiment, R⁵ is a monovalent saturated, substituted or unsubstituted, branched or straight chain hydrocarbyl moiety having from 1 to 15 carbon atoms, more suitably from 1 to 12 carbon atoms.

Some specific precursor compounds having an ester linkage and suitable for the above described embodiment of the present invention can be found in Table 1 below.

TABLE 1

| Compound | Formula |
|---|---|
| Benzyl (s)-(−)- lactate | 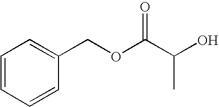 |
| Benzyl ethyl malonate | 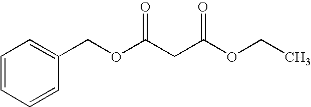 |
| Benzyl- laurate | 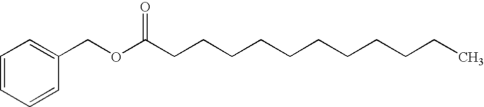 |
| Benzyl benzoate | 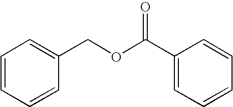 |
| Benzyl paraben | 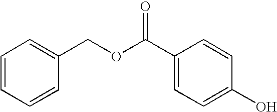 |
| Benzyl salicylate | 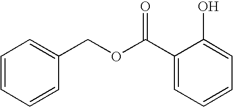 |
| Phenoxy- ethyl paraben | 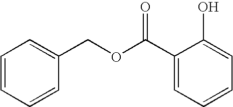 |

The above compounds can be used alone or in combinations thereof.

As noted herein, enzymes such as esterase produced by bacteria such as S. aureus found in the vaginal flora along with enzymes naturally occurring in the menstrual fluid, can hydrolyze the precursor compounds described herein to produce an active species and a second compound. For example, the enzyme esterase can react with the ester linkage of the precursor compounds described above to form the active species benzyl alcohol and a hydrocarbon. Benzyl alcohol has been found to substantially inhibit exoproteins from Gram positive bacteria without substantially eliminating the bacteria.

In another embodiment, the precursor compounds have the general chemical structure: 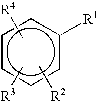
wherein R¹ is 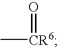
R⁶ is selected from the group consisting of an amino acid, a methyl ester of an amino acid, and an ethyl ester of an amino acid; R², R³, and R⁴ are independently selected from the group consisting of H, OH, COOH.

Amino acids are organic compounds containing an amino group and a carboxylic acid group. Suitable amino acids that can be used for R⁶ are any of the twenty amino acids found naturally in the human body. More particularly, amino acids for use in the present invention suitably include, for example, valine, leucine, cysteine, and combinations thereof.

Suitable compounds for this embodiment can be found in Table 2 below.

TABLE 2

| Compound | Formula |
|---|---|
| N-Benzoyl-DL-Valine | 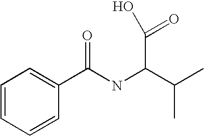 |
| N-Benzoyl-DL-Leucine | 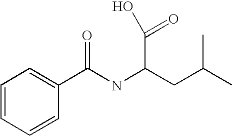 |
| N-Benzoyl-DL-Cysteine | 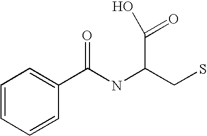 |

The above compounds described herein can be used alone or in combinations thereof.

As described above, enzymes such as amidase produced by bacteria such as S. aureus found in the vaginal flora along with enzymes naturally occurring in the menstrual fluid can hydrolyze the precursor compounds containing the amino acid. For example, the enzyme amidase can react with the precursor compound and break the amide bonds of the compounds in this embodiment to release benzoic acid and an amino acid. Like benzyl alcohol discussed above, benzoic acid has been found to substantially inhibit exoproteins from Gram positive bacteria without substantially eliminating the naturally occurring flora.

In accordance with the present invention, the non-absorbent article comprises an effective amount of the precursor compound such that upon hydrolysis of the precursor compound, there is a sufficient amount of active agent produced to substantially inhibit the formation of exoproteins such as TSST-1 when the non-absorbent article is exposed to S. aureus bacteria. Several methods are known in the art for testing the effectiveness of potential inhibitory agents, such as benzyl alcohol or benzoic acid on the inhibition of the production of TSST-1 in the presence of S. aureus. One such preferred method is set forth in Example 1. When tested in accordance with the methodology set forth herein, preferably, the active species produced from the hydrolysis of the precursor compound reduces the formation of TSST-1 by S. aureus by at least about 40%, more preferably by at least about 50%, still more preferably by at least about 60%, still more preferably by at least about 70%, still more preferably by at least about 80%, still more preferably by at least about 90%, and still more preferably by at least about 95%. Additionally, as noted above, the precursor compound may also inhibit the production of exoproteins from Gram positive bacteria in some embodiments. The test procedure set forth in Example 1 can also be used to measure the amount of inhibition by the precursor compound. In some embodiments, the precursor compound may reduce the formation of TSST-1 by at least about 50%, preferably at least about 80%.

In accordance with the present invention, the non-absorbent products comprise a suitable amount of precursor compound such that, upon use, the precursor compound and/or the active species produced therefrom in or around the vagina as discussed herein can substantially inhibit the production of exoprotein from Gram positive bacteria. Generally, non-absorbent products will comprise from about 0.15% (by weight of the non-absorbent substrate) to about 2.0% (by weight of the non-absorbent substrate) precursor compound. These percentages are commonly referred to as “add on weight percentages.” In a desirable embodiment, the non-absorbent products will comprise from about 0.17% (by weight of the non-absorbent substrate) to about 1.7% (by weight of the non-absorbent substrate) precursor compound.

In one specific embodiment, a tampon applicator as described herein comprises a suitable amount of precursor compound such that, upon use, the precursor compound and/or the active species produced therefrom in or around the vagina as discussed herein can substantially inhibit the production of exoprotein from Gram positive bacteria. Generally, the tampon applicator will comprise from about 0.15% (by weight of the non-absorbent substrate) to about 2.0% (by weight of the non-absorbent substrate) precursor compound. In a desirable embodiment, the tampon applicator will comprise from about 0.17% (by weight of the non-absorbent substrate) to about 1.7% (by weight of the non-absorbent substrate) precursor compound.

In a preferred embodiment of the present invention, the precursor compounds described herein can be introduced into and/or onto the non-absorbent article in combination with one or more surface-active agents to further reduce the production of exoproteins such as TSST-1 without significantly eliminating the beneficial bacterial flora. The surface-active agents used in combination without the precursor compounds may also act as lubricants and/or emollients to further improve product performance. In one embodiment including a tampon applicator, a suitable surface-active agent is myreth-3-myristate, which is commercially sold as CETIOL 1414 by Kraft Chemical Corp. (Melrose Park, Ill.). Other suitable surface-active agents for the present invention include, for example, glycerol monolaurate and laureth-4.

In accordance with the present invention, the non-absorbent products can comprise a suitable amount of surface-active agents such that, upon use, the surface-active agents can further inhibit the production of exoprotein from Gram positive bacteria. Generally, the non-absorbent products will comprise from about 0.4% (by weight of the non-absorbent substrate) to about 1.1% (by weight of the non-absorbent substrate) surface-active agent. In one specific embodiment, the non-absorbent product is a tampon applicator comprising about 0.75% (by weight of the non-absorbent substrate) surface-active agent.

Additionally, the precursor compounds described herein can be introduced into and/or onto the non-absorbent product in combination with one or more secondary agents to further reduce the production of exoproteins such as TSST-1 without significantly eliminating the beneficial bacterial flora. Suitable examples of secondary agents useful in the present invention include agents selected from the group consisting of: compounds with an ether, ester, amide, glycosidic, or amine bond linking a C₈-C₁₈ fatty acid to an aliphatic alcohol.

In one embodiment, the precursor compound described herein can be used in combination with ester compounds having the general formula: 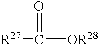
wherein R²⁷ is a straight or branched alkyl or straight or branched alkenyl having from 8 to about 18 carbon atoms and R²⁸ is selected from the group consisting of an alcohol, a polyhydric alcohol, and an ethoxylated alcohol. As used herein, the term “polyhydric” refers to the presence in a chemical compound of at least two hydroxyl (OH) groups. Suitable compounds include glyceryl monolaurate, glyceryl dilaurate, myreth-3-myristate, and mixtures thereof.

In another embodiment, the precursor compound described herein can be used in combination with ether compounds having the general formula:
R¹⁰—O—R¹¹
wherein R¹⁰ is a straight or branched alkyl or straight or branched alkenyl having from 8 to about 18 carbon atoms and R¹¹ is selected from the group consisting of an alcohol, an ethoxylated alcohol, a polyalkoxylated sulfate salt and a polyalkoxylated sulfosuccinate salt. Suitable compounds include laureth-3, laureth-4, laureth-5, PPG-5 lauryl ether, 1-O-dodecyl-rac-glycerol, sodium laureth sulfate, potassium laureth sulfate, disodium laureth (3) sulfosuccinate, dipotassium laureth (3) sulfosuccinate, and polyethylene oxide (2) sorbital ether.

In another embodiment, the precursor compounds described herein can be used in combination with an alkyl polyglycoside compound. Suitable alkyl polyglycosides for use in combination with the precursor compounds include alkyl polyglycosides having the general formula:
H—(Zn)—O—R¹⁴
wherein Z is a saccharide having 5 or 6 carbon atoms, n is a whole number from 1 to 6, and R¹⁴ is a linear or branched alkyl group having from about 8 to about 18 carbon atoms. Glucopon 220, 225, 425, 600, and 625 (all commercially available from Henkel Corporation, Ambler, Pa.) and TL 2141 (commercially available from ICI Surfactants, Wilmington, Del.) are suitable alkyl polyglycosides for use in combination with the precursor compounds of the present invention.

In another embodiment, the precursor compounds described herein can be used in combination with an amide containing compound having the general formula: 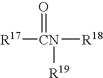
wherein R¹⁷, inclusive of the carbonyl carbon, is an alkyl group having 8 to 18 carbon atoms, and R¹⁸ and R¹⁹ are independently selected from hydrogen or an alkyl group having from 1 to about 12 carbon atoms which may or may not be substituted with groups selected from ester groups, ether groups, amine groups, hydroxyl groups, carboxyl groups, carboxyl salts, sulfonate groups, sulfonate salts, and mixtures thereof. Preferred amide compounds for use in combination with the precursor compounds described herein include sodium lauryl sarcosinate, lauramide monoethanolamide, lauramide diethanolamide, lauramidopropyl dimethylamine, disodium lauramido monoethanolamide sulfosuccinate, and disodium lauroamphodiacetate.

In another embodiment, the precursor compounds described herein can be used in combination with amine compounds having the general formula: 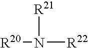
wherein R²⁰ is an alkyl group having from about 8 to about 18 carbon atoms and R²¹ and R²² are independently selected from the group consisting of hydrogen and alkyl groups having from 1 to about 18 carbon atoms and which can have one or more substitutional moieties selected from the group consisting of hydroxyl, carboxyl, carboxyl salts, and imidazoline. Preferred amine compounds for use with the precursor compounds described herein include triethanolamide laureth sulfate, lauramine, lauramino propionic acid, sodium lauriminodipropionic acid, lauryl hydroxyethyl imidazoline, and mixtures thereof.

In another embodiment, the amine compound can be an amine salt having the general formula: 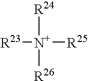
wherein R²³ is an anionic moiety associated with the amine and is derived from an alkyl group having from 8 to about 18 carbon atoms and R²⁴, R²⁵, and R²⁶ are independently selected from the group consisting of hydrogen and alkyl group having from 1 to about 18 carbon atoms and which can have one or more substitutional moieties selected from the group consisting of hydroxyl, carboxyl, carboxyl salts, and imidazoline. R²⁴, R²⁵, and R²⁶ can be saturated or unsaturated. A preferred compound illustrative of an amine salt is TEA laureth sulfate.

Amounts of secondary compounds described herein to be added to the non-absorbent products to further reduce the production of TSST-1 have been found to be from about 0.15% (by weight of the non-absorbent substrate) to about 2.0% (by weight of the non-absorbent substrate) secondary compound. More suitably, the non-absorbent products comprise from about 0.17% (by weight of the non-absorbent substrate) to about 1.7% (by weight of the non-absorbent substrate) secondary compound.

The precursor compounds may be applied to the non-absorbent substrate using conventional methods for applying a chemical agent to the desired non-absorbent substrate. For example, non-absorbent articles may be dipped directly into a liquid bath containing the precursor compound and then can be air dried, if necessary, to remove any volatile solvents. Alternatively, the non-absorbent articles of the present invention can be sprayed or otherwise coated with the inhibitory compounds of the present invention.

The precursor compounds may additionally employ one or more conventional pharmaceutically-acceptable and compatible carrier materials useful for the desired application. The carrier can be capable of co-dissolving or suspending the precursor compound used in the non-absorbent article. Carrier materials suitable for use in the instant invention include those well-known for use in the cosmetic and medical arts as a basis for ointments, lotions, creams, salves, aerosols, suppositories, gels, and the like. For example, one suitable carrier is Cetiol. Additionally, as discussed above, Cetiol can also act as both a lubricant and an emollient. Other suitable carrier materials include water, various alcohols, and other organic solvents.

In another embodiment, the precursor compounds of this invention can be formulated into a variety of formulations such as those in current commercial douche formulations, or in higher viscosity douches. For example, the precursor compounds of the present invention can be incorporated in formulations used to irrigate and cleanse the vagina and prevent vaginal infections. Additionally, the precursor compounds can be formulated with surfactants, desirably nonionic surfactants, such as Cremophos RH60, Tween 20 or the like. The formulations of this invention may also contain preservatives. Compounds that can impart greater viscosity, such as propylene glycol, may be added to the formulations of this invention. Generally, higher viscosity formulations are preferred in order to create formulations that will tend to remain in the vagina for a relatively long time period after administration.

When the precursor compounds are incorporated into a formulation, which includes a pharmaceutically acceptable carrier, the formulation typically contains at least about 0.01% (wt/vol) and desirably at least about 0.4% (wt/vol) precursor compound (based on the total weight of the formulation). Generally, the formulations contain no more than about 0.3% (wt/vol) precursor compound. Particularly suitable formulations for use in vaginal cleansing applications can contain from at least about 0.2 millimoles/liter to about 50 millimoles/liter, suitably from about 0.3 millimoles/liter to about 30 millimoles/liter and, more suitably, from about 1.0 millimole/liter to about 15 millimoles/liter of the precursor compound.

The precursor compounds of the present invention may, in some embodiments, be used in combinations with adjunct components conventionally found in pharmaceutical compositions in their art-established fashion and at concentrations that would not alter the normal vaginal flora. For example, the compositions may contain additional compatible pharmaceutically active materials for combination therapy, such as supplementary selective antibacterials, antioxidants, anti-parasitic agents, antipruritics, astringents, local anaesthetics, or anti-inflammatory agents.

In one embodiment, the precursor compounds can be microencapsulated in a shell-type material that will dissolve, disintegrate, rupture, or otherwise breakdown upon contact with menses or other vaginal secretions to release the component. With this embodiment, the encapsulation material retards volatilization of the precursor compound until wetted with a bodily secretion, which results in a release of the precursor compound. Such encapsulation can significantly increase the amount of precursor compound present in the product after manufacturing and storage. Suitable microencapsulated shell materials are known in the art and include cellulose-based polymeric materials (e.g., ethyl cellulose), carbohydrate-based materials (e.g., cationic starches and sugars) and materials derived therefrom (e.g., dextrins and cyclodextrins) as well as other materials compatible with human tissues.

The microencapsulation shell thickness may vary and is generally manufactured to allow the encapsulated precursor compound to be covered by a thin layer of encapsulation material, which may be a monolayer or thicker laminate layer, or may be a composite layer. The microencapsulation layer should be thick enough to resist cracking or breaking of the shell during handling or shipping of the product. The microencapsulation layer should be constructed such that humidity from atmospheric conditions during storage, shipment, or wear will not cause a breakdown of the microencapsulation layer and result in a release of the precursor compound.

Microencapsulated formulations or components applied directly to the non-absorbent articles should be of a size such that the user cannot feel the encapsulated shell on the skin or mucosa during use. Typically, the capsules have a diameter of no more than about 25 micrometers, and desirably no more than about 10 micrometers. At these sizes, there is no “gritty” or “scratchy” feeling when the formulation contacts the skin.

The present invention is illustrated by the following examples, which are merely for the purpose of illustration and are not to be regarded as limiting the scope of the invention or manner in which it may be practiced.

### EXAMPLE 1

In this Example, the effect of various test compounds on the growth of S. aureus and production of TSST-1 was determined. The test compound, in the desired concentration (expressed in wt % (w/v)) was placed in 10 mL of a growth medium in a sterile, 50 mL conical polypropylene tube (Sarstedt, Inc., Newton, N.C.).

The growth medium was prepared by dissolving 37 grams of brain heart infusion broth (BHI) (Difco Laboratories, Cockeysville, Md.) in 880 mL distilled water and sterilizing the broth according to the manufacturer's instructions. The BHI was supplemented with 100 mL fetal bovine serum (FBS) (Sigma Chemical Company, St. Louis, Mo.). Ten mL of a 0.021 M sterile solution of hexahydrate of magnesium chloride (Sigma Chemical Company, St. Louis, Mo.) was added to the BHI-FBS mixture. Ten mL of a 0.027 M sterile solution of L-glutamine (Sigma Chemical Company, St. Louis, Mo.) was also added to the BHI-FBS mixture.

Compounds to be tested included N-benzoyl-DL-leucine (Sigma B-1504) and N-benzoyl-DL-valine (Sigma B-6500). Test compounds were received as solids. The solids were dissolved in BHI prepared as described above. The test compounds were added to the growth medium in the amount necessary to obtain the desired final concentration. Cetiol 1414E (myreth-3-myristate) (Kraft Chemical Corp., Melrose Park, Ill.) was included in the growth medium in some assays at a 10 mM concentration.

In preparation for inoculation of the tubes of growth medium containing the test compounds, an inoculating broth was prepared as follows: S. aureus MN8 was streaked onto a tryptic soy agar plate (TSA; Difco Laboratories, Cockeysville, Md.) and incubated at 35° C. The test organism in this example was obtained from Dr. Pat Schlievert, Department of Microbiology, University of Minnesota Medical School, Minneapolis, Minn. After 24 hours of incubation three to five individual colonies were picked with a sterile inoculating loop and used to inoculate 10 mL of growth medium. The tube of inoculated growth medium was incubated at 35° C. in atmospheric air. After 24 hours of incubation, the culture was removed from the incubator and mixed well on a S/P brand vortex mixer. A second tube containing 10 mL of the growth medium was inoculated with 0.5 mL of the above-described 24 hour old culture and incubated at 35° C. in atmospheric air. After 24 hours of incubation the culture was removed from the incubator and mixed well on a S/P brand vortex mixer. The optical density of the culture fluid was determined in a microplate reader (Bio-Tek Instruments, Model EL309, Winooski, Vt.). The amount of inoculum necessary to give 5×10⁶ CFU/mL in 10 mL of growth medium was determined using a previously prepared standard curve.

This Example included tubes of growth medium with varying concentrations of test compounds, varying concentrations of test compounds and Cetiol 1414E, and tubes of growth medium without test compounds (control). Each tube was inoculated with the amount of inoculum determined as described above. The tubes were capped with foam plugs (IDENTI-PLUG plastic foam plugs, Jaece Industries, purchased from VWR Scientific Products, South Plainfield, N.J.). The tubes were incubated at 35° C. in atmospheric air containing 5% by volume CO₂. After 24 hours of incubation the tubes were removed from the incubator, the culture fluid was assayed for the number of colony forming units of S. aureus, and the culture fluid was prepared for the analysis of TSST-1 as described below.

The number of colony forming units per mL after incubation was determined using standard plate count procedures. In preparation for analysis of TSST-1, the culture fluid broth was centrifuged at 2500 rpm at 2-10° C. for 15 minutes and the supernatant was subsequently filter sterilized through a FISHERBRAND 0.45 μm MCE filter, 0.2 μM pore size. The resulting fluid was frozen at −70° C. in a FISHERBRAND 12×75 mm polystyrene culture tube (Fisher Scientific, Pittsburgh, Pa.).

The amount of TSST-1 per mL was determined by a non-competitive, sandwich enzyme-linked immunoabsorbent assay (ELISA). The method employed was as follows: four reagents, TSST-1 (#TT-606), rabbit polyclonal anti-TSST-1 IgG (LTI-101), rabbit polyclonal anti-TSST-1 IgG conjugated to horseradish peroxidase (#LTC-101), and normal rabbit serum (NRS) certified anti-TSST-1 free (#NRS-10) were purchased from Toxin Technology, Inc. (Sarasota, Fla.). A 10 μg/mL solution of the polyclonal rabbit anti-TSST-1 IgG was prepared in phosphate buffered saline (PBS) (pH 7.4). The PBS was prepared from 0.016 M NaH₂PO₄, 0.004 M NaH₂PO₄—H₂O, 0.003 M KCl and 0.137 M NaCl, all available from Sigma Chemical Company (St. Louis, Mo.). One hundred microliters of the polyclonal rabbit anti-TSST-1 IgG solution was pipetted into the inner wells of polystyrene microplates, (Nunc-Denmark, Catalogue Number #439454). The plates were covered and incubated at room temperature overnight. Unbound anti-toxin was removed by draining until dry.

TSST-1 was diluted to 10 ng/mL with phosphate buffered saline (PBS) (pH 7.4) containing 0.05% (vol/vol) Tween-20 (PBS-Tween) (Sigma Chemical Company, St. Louis, Mo.) and 1% (vol/vol) NRS and incubated at 4° C. overnight. Test samples were combined with 1% NRS (vol/vol) and incubated at 4° C. overnight. Samples of the culture fluid and the TSST-1 reference standard were assayed in triplicate.

One hundred microliters of a 1% (wt/vol) solution of the sodium salt of casein (Sigma Chemical Company, St. Louis, Mo.) in PBS were pipetted into the inner wells of polystyrene microplates. The plates were covered and incubated at 35° C. for one hour. Unbound BSA was removed by 3 washes with PBS-Tween. TSST-1 reference standard (10 ng/mL) treated with NRS, test samples treated with NRS, and reagent controls were pipetted in 200 μL volumes to their respective wells on the first and seventh columns of the plate. One hundred microliters of PBS-Tween were added to the remaining wells. The TSST-1 reference standard and test samples were then serially diluted 5 times in the PBS-Tween by transferring 100 microliters from well-to-well. The samples were mixed prior to transfer by repeated aspiration and expression. Samples of the test samples and the TSST-1 reference standard were assayed in triplicate. This was followed by incubation for 1.5 hours at 35° C. and five washes with PBS-T and three washes with distilled water to remove unbound toxin. The rabbit polyclonal anti-TSST-1 IgG conjugated to horseradish peroxidase was diluted according to manufacturer's instructions and 50 microliters was added to each microtiter well, except well A-1, the conjugate control well. The plates were covered and incubated at 35° C. for one hour.

Following incubation the plates were washed five times in PBS-Tween and three times with distilled water. Following the washes, the wells were treated with 100 microliters of a horseradish peroxidase substrate buffer consisting of 5 mg of o-phenylenediamine and 5 μL of 30% hydrogen peroxide (both available from Sigma Chemical Company, St. Louis, Mo.) in 11 mL of citrate buffer (pH 5.5). The citrate buffer was prepared from 0.012 M anhydrous citric acid and 0.026 M dibasic sodium phosphate both available from Sigma Chemical Company (St. Louis, Mo.). The plates were incubated for 15 minutes at 35° C. The reaction was stopped by the addition of 50 microliters of a 5% sulfuric acid solution. The intensity of the color reaction in each well was evaluated using the Bio-Tek Model EL309 microplate reader (OD 490 nm). TSST-1 concentrations in test samples were determined from the reference toxin regression equation derived during each assay procedure. The efficacy of the compound in inhibiting the production of TSST-1 is shown in Table 3 below.

TABLE 3

| | Amount | | | | |
|---|---|---|---|---|---|
| Amino acid | test | | | ELISA: TSST-1 | |
| containing | compound | Cetiol | | ng per mL | |
| compound | (wt %) | 1414E | CFU/mL | (% inhibition) | |
| None | | None | 2.63E+08 | 178.4 | (N/A) |
| N-benzoyl-DL- | 0.25% | None | 2.69E+08 | 14.6 | (92.8%) |
| leucine | (w/v) | | | | |
| N-benzoyl-DL- | 0.20% | None | 1.61E+08 | 25.1 | (79%) |
| valine | (w/v) | | | | |
| None | | 10 mM | 1.75E+08 | 53.1 | (60%) |
| N-benzoyl-DL- | 0.25% | 10 mM | 2.23E+08 | 7.9 | (95%) |
| leucine | (w/v) | | | | |
| N-benzoyl-DL- | 0.20% | 10 mM | 2.07E+08 | 10.6 | (93.2%) |
| valine | (w/v) | | | | |

N/A = Not Applicable

In accordance with the present invention, the data in Table 3 shows that S. aureus MN8, when compared to the control, produced less TSST-1 in the presence of the amino acid containing test compounds. At the concentrations tested, these compounds reduced the amount of toxin produced by 79% to 93%. Also the data shows that S. aureus MN8, when compared to the control, produced less TSST-1 in the presence of the amino acid containing test compounds when combined with Cetiol 1414E. At the concentrations tested, these compounds, when combined with Cetiol 1414E, reduced the amount of toxin produced by 93% to 95%. However, although the amount of toxin produced was significantly reduced, there was minimal, if any, effect on the growth of S. aureus.

### EXAMPLE 2

In this Example, the effect of benzyl alcohol (Aldrich 40,283-4) and benzyl ethyl malonate (Aldrich 30,069-1) (Sigma Chemical Corporation, St. Louis, Mo.) on the growth of S. aureus and the production of TSST-1 was determined. The effect of the test compounds tested in Example 2 was determined by placing the desired concentration, expressed in % (v/v), in 10 mL of a growth medium as described in Example 1. The test compounds were then tested and evaluated as in Example 1, except that each test was carried out in quadruplicate. The results shown represent an average of the four values. The effect of the test compounds on growth of S. aureus MN8 and on the production of TSST-1 is shown in Table 4 below.

TABLE 4

| | Amount | | | | |
|---|---|---|---|---|---|
| | Test | | | ELISA: TSST-1 | |
| | Compound | Cetiol | | ng per mL | |
| Compound | (%(v/v)) | 1414E | CFU/mL | (% inhibition) | |
| None | | None | 7.36E+08 | 338 | (N/A) |
| Benzyl alcohol | 0.3% | None | 7.52E+08 | 50 | (85%) |
| Benzyl ethyl | 0.8% | None | 3.78E+08 | 19 | (94%) |
| malonate | | | | | |
| None | | 10 mM | 2.61E+08 | 70 | (79%) |
| Benzyl alcohol | 0.3% | 10 mM | 5.02E+08 | 4 | (99%) |
| Benzyl ethyl | 0.8% | 10 mM | 1.51E+08 | 6 | (98%) |
| malonate | | | | | |

N/A = Not Applicable

At the concentrations tested, both the compound, benzyl ethyl malonate, and the active species, benzyl alcohol, reduced the amount of toxin produced by 85% to 94%. Also, the data shows that S. aureus MN8, when compared to the control, produced less TSST-1 in the presence of benzyl alcohol or benzyl ethyl malonate when combined with Cetiol 1414E (myreth-3-myristate). At the concentrations tested, these compounds, when combined with Cetiol 1414E, reduced the amount of toxin produced by 98% to 99%.

Statistical analysis of the treatments was performed using pairwise comparison on Least Squares Means in an Analysis of Variance context. The pairwise comparisons were equivalents of standard T Tests. The results of the comparisons showed that growth in the presence of Cetiol alone resulted in significantly greater inhibition of the growth of S. aureus than growth in its absence or growth in the presence of benzyl alcohol alone or benzyl ethyl malonate with or without Cetiol. Growth in the presence of benzyl ethyl malonate and the combinations of Cetiol with benzyl alcohol or benzyl ethyl malonate resulted in significantly decreased toxin production when compared to growth in the presence of Cetiol alone or no additives. However, although the amount of toxin produced was significantly reduced under these conditions, there was minimal, if any, effect on the growth of S. aureus.

### EXAMPLE 3

In this Example, the effect of benzyl (s)-(−)-lactate (Aldrich 42,484-6) (Sigma Chemical Corporation, St. Louis, Mo.) on the growth of S. aureus and the production of TSST-1 was determined. The effect of the test compounds was determined by placing the desired concentration, expressed in % (v/v), in 10 mL of a growth medium as in Example 1. The compounds were then tested and evaluated as in Example 1, except that each test was carried out in quadruplicate. The results shown represent an average of the four values. The effect of the test compounds on the growth of S. aureus MN8 and on the production of TSST-1 is shown in Table 5 below.

TABLE 5

| | Amount | | | | |
|---|---|---|---|---|---|
| | Test | | | ELISA: TSST-1 | |
| | Compound | Cetiol | | Nanograms/mL | |
| Compound | (%(v/v)) | 1414E | CFU/mL | (% inhibition) | |
| None | | None | 4.60E+08 | 604 | (N/A) |
| Benzyl (s)- | 0.3% | None | 4.86E+08 | 18 | (96.9%) |
| (−)-lactate | | | | | |
| None | | 10 mM | 2.48E+08 | 61 | (89.9%) |
| Benzyl (s)- | 0.3% | 10 mM | 1.48E+08 | 5 | (99.2%) |
| (−)-lactate | | | | | |

N/A = Not Applicable

At the concentration tested, benzyl (s)-(−)-lactate reduced the amount of toxin produced by 97%. Also, the data shows that S. aureus MN8, when compared to the control, produced less TSST-1 in the presence of benzyl (s)-(−)-lactate when combined with Cetiol 1414E (myreth-3-myristate). At the concentration tested, benzyl (s)-(−)-lactate, when combined with Cetiol 1414E, reduced the amount of toxin produced by 99%.

Statistical comparisons of the treatments were performed by the method discussed in Example 2. The results of the comparisons showed that growth in the presence of Cetiol alone or Cetiol and benzyl (s)-(−)-lactate resulted in significantly greater inhibition of the growth of S. aureus than growth in the absence of Cetiol or growth in the presence of benzyl (s)-(−)-lactate alone. Growth in the presence of benzyl (s)-(−)-lactate and the combinations of Cetiol with benzyl (s)-(−)-lactate resulted in significantly decreased toxin production when compared to growth in the presence of Cetiol alone or no additives.

### EXAMPLE 4

In this Example, the effect of benzyl (s)-(−)-lactate in combination with the surface-active agent Cetiol 1414E (myreth-3-myristate) was tested using a 5×4 checkerboard experimental design. This allowed the evaluation of the interaction of the two test compounds on the growth of S. aureus and the production of TSST-1.

Five concentrations of benzyl (s)-(−)-lactate (0.00, 0.06%, 0.13%, 0.25%, and 0.50%) were combined with four concentrations of Cetiol 1414E (10 mM, 5 mM, 2.5 mM, and 0.0 mM) in a twenty tube array. For example, tube #1 contained 0.0 mM Cetiol 1414E and 0.0% benzyl (s)-(−)-lactate (w/v) in 10 mL of growth medium (as prepared in Example 1). Each of the tubes #1-#20 contained a unique combination of benzyl (s)-(−)-lactate and Cetiol. The solutions were tested and evaluated as in Example 2. The effect of the test compounds on the growth of S. aureus MN8 and on the production of TSST-1 is shown in Table 6 below.

TABLE 6

| | Benzyl | | nag | ng TSST- | |
|---|---|---|---|---|---|
| Cetiol | (s)-(−)- | CFU/mL | TSST-1 | 1 per | % |
| mM | Lactate % | ×10⁷ | per mL | 10⁸ CFU | Reduction |
| 0 | 0 | 15.3 | 390 | 255 | N/A |
| 0 | 0.5 | 20.0 | 49 | 24 | 90% |
| 0 | 0.25 | 24.6 | 24 | 10 | 96% |
| 0 | 0.13 | 18.0 | 66 | 37 | 86% |
| 0 | 0.06 | 18.3 | 124 | 68 | 73% |
| 2.5 | 0 | 22.5 | 148 | 66 | 74% |
| 2.5 | 0.5 | 1.5 | 3 | 19 | 93% |
| 2.5 | 0.25 | 15.4 | 8 | 5 | 98% |
| 2.5 | 0.13 | 26.2 | 47 | 18 | 93% |
| 2.5 | 0.06 | 20.4 | 51 | 25 | 90% |
| 5.0 | 0 | 25.6 | 203 | 79 | 69% |
| 5.0 | 0.5 | 2.30 | 2 | 7 | 97% |
| 5.0 | 0.25 | 12.0 | 7 | 6 | 98% |
| 5.0 | 0.13 | 18.3 | 19 | 11 | 96% |
| 5.0 | 0.06 | 26.2 | 28 | 11 | 96% |
| 10.0 | 0 | 17.4 | 57 | 33 | 87% |
| 10.0 | 0.5 | 6.1 | 2 | 4 | 99% |
| 10.0 | 0.25 | 14.7 | 8 | 6 | 98% |
| 10.0 | 0.13 | 20.0 | 16 | 8 | 97% |
| 10.0 | 0.06 | 22.6 | 41 | 18 | 93% |

N/A = Not Applicable

At every concentration of Cetiol 1414E, benzyl (s)-(−)-lactate increases the inhibition of TSST-1 production. The effect appears to be additive.

### EXAMPLE 5

In this Example, the effect of benzyl laurate (Penta Manufacturing, Fairfield, N.J.) on the growth of S. aureus MN8 and the production of TSST-1 was determined. The effect of the test compound was determined by placing the desired concentration, expressed in % (v/v), in 10 mL of a growth medium as in Example 1. The compounds were then tested and evaluated as in Example 1. The effect of benzyl laurate on growth of S. aureus MN8 and on the production of TSST-1 is shown in Table 7 below.

TABLE 7

| | Amount | | | | |
|---|---|---|---|---|---|
| | Test | | | ELISA: TSST-1 | |
| | Compound | Cetiol | | ng per mL | |
| Compound | (%(v/v)) | 1414E | CFU/mL | (% inhibition) | |
| None | | None | 4.10E+08 | 450 | (N/A) |
| Benzyl laurate | 0.4% | None | 4.05E+08 | 225 | (62%) |
| Benzyl laurate | 0.6% | None | 3.80E+08 | 55 | (91%) |
| None | | 10 mM | 2.05E+08 | 153 | (79%) |
| Benzyl laurate | 0.4% | 10 mM | 2.05E+08 | 60 | (90%) |
| Benzyl laurate | 0.6% | 10 mM | 3.05E+08 | 83 | (86%) |

N/A = Not Applicable

At the concentrations tested, benzyl laurate reduced the amount of toxin produced by 62% to 91%. At the concentrations tested, benzyl laurate, when combined with Cetiol 1414E (myreth-3-myristate), reduced the amount of toxin produced by 86% to 90%.

### EXAMPLE 6

In this Example, commercial tampons (KOTEX super absorbency, Kimberly-Clark Worldwide, Inc., Neenah, Wis.) were treated with benzyl alcohol to determine the effect of the treated tampon on the growth of S. aureus MN8 and the production of TSST-1. As stated above, the commercial tampon will contain about 0.75% (by weight of the total tampon having a cover) Cetiol 1414E (myreth-3-myristate). In preparation for the experiment, the string of the tampon was cut off and the pledget was placed into a sterile, capped 50 mL polystyrene test tube with the string end down.

The pledgets were inoculated with 5 mL of five concentrations (0.0, 0.3%, 0.15%, 0.075%, and 0.03%) of benzyl alcohol dissolved in Brain Heart Infusion Broth (BHI). The tampon pledgets were left to sit at room temperature for one hour.

Each pledget was then inoculated with 5.5 mL of an inoculating broth containing 5×10⁶±1×10⁶ CFU/mL of S. aureus MN8 to achieve a final volume of 10.5 mL. The tubes were capped with foam plugs (IDENTI-PLUG plastic foam plugs, Jaece Industries, purchased from VWR Scientific Products, South Plainfield, N.J.) and incubated at 37° C. for 24 hours. The pledgets were removed from the incubator and individually placed into sterile STOMACHER bags (Seward Ltd., Norfolk, United Kingdom), which contained 50 mL sterile BHI. The pledgets and fluid were then stomached or blended in the bags. Aliquots of fluid were removed from the STOMACHER bags and placed into sterile tubes for testing.

Plate count samples were prepared by vortexing the sample, withdrawing 5 mL of the sample and placing the 5 mL in a new sterile 50 mL centrifuge tube. The sample was then sonicated using a Virsonic 600 Ultrasonic Cell Disruptor (Virtis Company, Gardiner, N.Y.) for 15 seconds at 8% output power. When all the samples had been sonicated, the number of colony forming units (CFU) per mL was determined using standard plate count procedures.

In preparation for analysis of TSST-1, the culture fluid broth was centrifuged at 9000 rpm at 4° C. for 5 minutes and the supernatant was subsequently filter sterilized through 0.45 μm MCE filter, 0.2 μM pore size. The resulting fluid was frozen at −70° C. in two 1 mL aliquots in 1.5 mL polypropylene screw cap freezer vials.

The amount of TSST-1 per mL was determined by a non-competitive, sandwich enzyme-linked immunoabsorbent assay (ELISA). The method employed was as follows: four reagents, TSST-1 (#606), rabbit polyclonal anti-TSST-1 IgG (LTI-101), rabbit polyclonal anti-TSST-1 IgG conjugated to horseradish peroxidase (#LTC-101), and normal rabbit serum (NRS) certified anti-TSST-1 free (#NRS-10) were purchased from Toxin Technology, Inc. (Sarasota, Fla.). Sixty-two microliters of #LTI-101 was diluted so that a 1:100 dilution gave an absorbance of 0.4 at 205 nm and subsequently added to 6.5 mL of Na₂CO₃ buffer, pH 7.2, 0.5 M carbonate buffer, pH 9.6, and 100 μL of the solution was pipetted into each of the inner wells of the polystyrene microplates (available from Nunc-Denmark, Catalogue Number #439454). The plates were covered and incubated at 37° C. overnight.

The unbound anti-toxin was removed by four washes in an automatic plate washer with phosphate buffered saline (0.016 M Na₂HPO₄, available from Sigma Chemical Co., St. Louis, Mo.), pH 7.2, and 0.9% (w/v) NaCl (VWR Scientific Products, South Plainfield, N.J.) containing 0.5% (v/v) Tween 20 (Sigma Chemical Co. St. Louis, Mo.). The plates were treated with 100 μL of a 1% (w/v) solution of bovine serum albumin (BSA) fraction V (Sigma Chemical Co., St. Louis, Mo.), in the Na₂CO₃ plus NaHCO₃ buffer described above. The plates were again covered and incubated at 37° C. for one hour. Unbound BSA was removed by six washes of 250 μL PBS-Tween.

The test samples were then treated with normal rabbit serum (10% (v/v) concentration) for 15 minutes at room temperature. The TSST-1 reference standard (serially diluted from 2-20 ng/mL in PBS-Tween) and the NRS treated test samples (serially diluted in PBS-Tween so that the resultant TSST-1 concentration is between 2-20 ng), were pipetted in 100 μL volumes to their respective wells. The samples were then incubated for two hours at 37° C. and unbound toxins were then removed with four washes of 250 μL PBS-Tween. The rabbit polyclonal anti-TSST-1 IgG conjugated to horseradish peroxidase was diluted according to the manufacturer's instructions. The final use dilution of the conjugate was determined by running standard curves of TSST-1 reference standard with the conjugate at undiluted, 1:2, and 1:4 dilutions. The dilution that gave results most comparable to previous lots of conjugate was selected. One hundred μL volumes of this dilution were added to each microtiter well. The plates were covered and incubated at 37° C. for one hour.

Following incubation, the plates were washed six times in 250 μL PBS-Tween. Following the washes, the wells were treated with 100 μL of a horseradish peroxidase substrate solution consisting of 0.015 M sodium citrate, pH 4.0, 0.6 mM 2,2′-Azino-bis-(3-ethylbenzthiazoline-6-sulfonic acid) diammonium salt and 0.009% (v/v) hydrogen peroxide (all available from Sigma Chemical Co., St. Louis, Mo.). The intensity of the color reaction in each well was evaluated over time using a VersaMax Molecular Devices Microplate reader (OD 405 nm) and SoftMax Pro software (both available from Molecular Devices, Inc.). TSST-1 concentrations in the test samples were derived from the reference toxin regression equations for each assay procedure.

The efficacy of benzyl alcohol in inhibiting the production of TSST-1 by S. aureus is shown in Table 8 below.

TABLE 8

| Benzyl alcohol | | TSST-1 ng/mL | |
|---|---|---|---|
| add-on (wt %) | CFU/mL (×10⁸) | (% inhibition) | |
| 0* | 8.17 | 2.66 | (N/A) |
| 0.03% | 6.13 | 2.46 | (8%) |
| 0.075% | 2.83 | 2.22 | (17%) |
| 0.15% | 0.5 | 1.33 | (50%) |

N/A = Not Applicable
*As stated above, this Example uses commercial tampons. As such, this sample contains about 0.75% (by weight of tampon with cover) Cetiol 1414E (myreth-3-myristate).

In accordance with the present invention, the data shows that S. aureus MN8 produced less TSST-1 in the presence of tampons that contain both benzyl alcohol and Cetiol 1414E (myreth-3-myristate) as compared to the control tampons that contain only Cetiol 1414E. At the concentrations tested, benzyl alcohol reduced the amount of toxin produced by 8% to 50%.

### EXAMPLE 7

In this Example, the effect of the growth of S. aureus MN8 on the integrity of various test compounds was determined by measuring the amount of breakdown of the test compounds caused by the enzymes produced by S. aureus MN8 bacteria. The test compounds, in the desired concentrations, were placed in 100 mL of a growth medium in a sterile, 500 mL Corning Fleaker (Fisher Scientific, Pittsbury, Pa.). The growth medium and inoculum were prepared as in Example 1.

Compounds to be tested included 0.2% (v/v) benzyl alcohol (Aldrich 40,283-4), 0.5% (wt/v) sodium benzoate, 0.3% (v/v) benzyl (s)-(−)-lactate (Aldrich 42,484-6), 0.8% (v/v) benzyl ethyl malonate (Aldrich 30,069-1), and 0.3% (wt/v) N-benzoyl-DL-leucine (Sigma B-1504). The test compounds were added to the growth medium in the amount necessary to obtain the desired final concentration.

Each fleaker was inoculated with the amount of inoculum determined as described above. The fleakers were capped with sterile aluminum foil and incubated at 35° C. in atmospheric air in a Lab-Line orbital water bath (available from VWR Scientific Products, McGaw Park, Ill.) at 180 rpm. Fifteen milliliter samples were removed at 3, 6, 9, and 24 hours. The optical density (595 nm) of the culture fluid was determined and the culture fluid collected at 24 hours was assayed for the number of colony forming units of S. aureus MN8 using standard plate count procedures. The test compounds, at the concentrations tested, did not inhibit the growth of S. aureus.

In preparation for analysis of the integrity of the test compounds, the culture fluid was centrifuged at 3000 rpm at 2-10° C. for 15 minutes. The supernatant was filter sterilized through an AUTOVIAL 5 syringeless filter, 0.45 μM pore size (available from Whatman, Inc., Clifton, N.J.). The resulting fluid was frozen at −70° C. in a FISHERBRAND (12 mm×75 mm) polystyrene culture tube (Fisher Scientific, Pittsburgh, Pa.) until chemical analysis could be performed.

Using an Agilent Technologies 5973N GC/MS, analysis was performed on undiluted solutions at 3, 6, 9, and 24 hours to evaluate the ability of S. aureus MN8 to breakdown the test compounds. Based on the analysis, the control sample, which had no test compounds added, comprised mostly acetic acid at all time intervals. The samples containing benzoic acid and benzyl alcohol were found to have benzoic acid and benzyl alcohol respectively, as the dominant compound, but the benzoic acid and benzyl alcohol concentrations decreased over time. The sample comprising N-benzoyl-DL-leucine as the test compound was found to contain benzoic acid as the dominant compound, having a decreasing concentration over time. The sample comprising benzyl (s)-(−)-lactate as the test compound was found to contain benzyl alcohol as the dominant compound. Further, it was found that the concentration of benzyl alcohol increased over time. Finally, the sample comprising benzyl ethyl malonate as the test compound was found to contain benzyl alcohol as the dominant compound. Further, it was found that the concentration of benzyl alcohol increased over time.

In accordance with the present invention, the GC/MS analysis above show that the precursor compounds were broken down by the enzymes produced by S. aureus MN8 to produce the active species. It can further be seen that the precursor compounds were slowly broken down over time to allow for a long-lasting continous inhibition of exoprotein production by the active species.

Additional analysis of the 6 hour and 24 hour samples was conducted by liquid chromatography. In preparation for the chromatography, the samples were diluted 10 fold with water. The dilutions were then analyzed using an ion exclusion column to achieve the separation. Detection of the compounds was accomplished through UV absorbance at 230 nm.

In accordance with the present invention, the liquid chromatography analysis of the test compounds showed that the compounds were broken down into the active species, benzoic acid and benzyl alcohol, over a period of 24 hours. Specifically, the 6 hour and 24 hour samples containing 0.3% N-benzoyl-DL-leucine showed evidence of the compound's breakdown to benzoic acid. Additionally, the 6 hour and 24 hour samples containing benzyl (s)-(−)-lactate and benzyl ethyl malonate showed evidence of the compounds' breakdown to benzyl alcohol. As can further be seen, the 24 hour samples containing benzyl (s)-(−)-lactate and benzyl ethyl malonate showed evidence of an elevated level of benzyl alcohol compared to the 6 hour samples.

In view of the above, it will be seen that the several objects of the invention are achieved and other advantageous results obtained.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above without departing from the scope of the invention, it is intended that all matter contained in the above description and shown in the accompanying drawings shall be interpreted as illustrative and not in a limiting sense.

What is claimed is:

1. An exoprotein inhibitor for inhibiting the production of exoprotein from Gram positive bacteria comprising a non-absorbent substrate suitable for insertion into the vagina, the non-absorbent substrate having deposited thereon an effective amount of a precursor compound having the general formula: 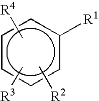
wherein R¹ is selected from the group consisting of 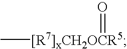
R⁷ is —OCH₂—; X is 0 or 1; R⁵ is a substituted or unsubstituted aromatic ring or a monovalent saturated or unsaturated, substituted or unsubstituted, branched or straight chain hydrocarbyl moiety that may or may not be substituted with hetero atoms; R², R³, and R⁴ are independently selected from the group consisting of H, OH, COOH, wherein upon hydrolysis the precursor compound is capable of producing an active species effective in inhibiting the production of exoprotein from Gram positive bacteria.

2. The exoprotein inhibitor as set forth in claim 1 wherein R⁵ is a monovalent saturated, substituted or unsubstituted, branched or straight chain hydrocarbyl moiety that may or may not be substituted with hetero atoms having from 1 to 15 carbon atoms.

3. The exoprotein inhibitor as set forth in claim 1 wherein R⁵ is a monovalent saturated, substituted or unsubstituted, branched or straight chain hydrocarbyl moiety that may or may not be substituted with hetero atoms having from 1 to 12 carbon atoms.

4. The exoprotein inhibitor as set forth in claim 1 wherein R² is selected from the group consisting of H and OH, and R³ and R⁴ are independently H.

5. The exoprotein inhibitor as set forth in claim 1 further comprising a surface-active agent selected from the group consisting of myreth-3-myristate, glycerol monolaurate, and laureth-4.

6. The exoprotein inhibitor as set forth in claim 1 wherein the precursor compound is selected from the group consisting of benzyl(s)-(−)-lactate, benzyl ethyl malonate, benzyl-laurate, benzyl benzoate, benzyl paraben, and benzyl salicylate.

7. The exoprotein inhibitor as set forth in claim 6 wherein the precursor compound is benzyl(s)-(−)-lactate.

8. The exoprotein inhibitor as set forth in claim 7 further comprising a surface-active agent selected from the group consisting of myreth-3-myristate, glycerol monolaurate, and laureth-4.

9. The exoprotein inhibitor as set forth in claim 6 wherein the precursor compound is benzyl ethyl malonate.

10. The exoprotein inhibitor as set forth in claim 9 further comprising a surface-active agent selected from the group consisting of myreth-3-myristate, glycerol monolaurate, and laureth-4.

11. The exoprotein inhibitor as set forth in claim 1 wherein the precursor compound is present in an amount of from about 0.15% (by weight of the non-absorbent substrate) to about 2.0% (by weight of the non-absorbent substrate).

12. The exoprotein inhibitor as set forth in claim 1 wherein the precursor compound is present in an amount of from about 0.17% (by weight of the non-absorbent substrate) to about 1.7% (by weight of the non-absorbent substrate)

13. The exoprotein inhibitor as set forth in claim 1 wherein the precursor compound is microencapsulated in a shell material.

14. The exoprotein inhibitor as set forth in claim 13 wherein the shell material comprises a material selected from the group consisting of cellulose-based polymeric materials, carbohydrate-based materials, and materials derived therefrom.

15. The exoprotein inhibitor as set forth in claim 1 further comprising a pharmaceutically active material selected from the group consisting of selective antibacterials, antioxidants, anti-parasitic agents, antipruritics, astringents, local anaesthetics, and anti-inflammatory agents.

16. The exoprotein inhibitor as set forth in claim 1 wherein the non-absorbent substrate is selected from the group consisting of a non-absorbent incontinence device, a barrier birth control device, a non-absorbent contraceptive device, a tampon applicator, and a douche.

17. An exoprotein inhibitor for inhibiting the production of exoprotein from Gram positive bacteria comprising a non-absorbent substrate suitable for insertion into the vagina, the non-absorbent substrate having deposited thereon an effective amount of a precursor compound having the general formula: 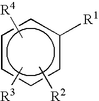
wherein R¹ is 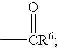
R⁶ is selected from the group consisting of an amino acid, a methyl ester of an amino acid, and an ethyl ester of an amino acid; R², R³, and R⁴ are independently selected from the group consisting of H, OH, COOH, wherein upon hydrolysis the precursor compound is capable of producing an active species effective in inhibiting the production of exoprotein from Gram positive bacteria.

18. The exoprotein inhibitor as set forth in claim 17 wherein R⁶ is an amino acid.

19. The exoprotein inhibitor as set forth in claim 18 wherein the amino acid is selected from the group consisting of valine, leucine, and cysteine.

20. The exoprotein inhibitor as set forth in claim 17 further comprising a surface-active agent selected from the group consisting of myreth-3-myristate, glycerol monolaurate, and laureth-4.

21. The exoprotein inhibitor as set forth in claim 17 wherein the precursor compound is selected from the group consisting of n-benzoyl-dl-valine, n-benzoyl-dl-leucine, and n-benzoyl-dl-cysteine.

22. The exoprotein inhibitor as set forth in claim 21 further comprising a surface-active agent selected from the group consisting of myreth-3-myristate, glycerol monolaurate, and laureth-4.

23. The exoprotein inhibitor as set forth in claim 17 wherein the precursor compound is present in an amount of from about 0.15% (by weight of the non-absorbent substrate) to about 2.0% (by weight of the non-absorbent substrate).

24. The exoprotein inhibitor as set forth in claim 17 wherein the precursor compound is present in an amount of from about 0.17% (by weight of the non-absorbent substrate) to about 1.7% (by weight of the non-absorbent substrate).

25. The exoprotein inhibitor as set forth in claim 17 wherein the precursor compound is microencapsulated in a shell material.

26. The exoprotein inhibitor as set forth in claim 25 wherein the shell material comprises a material selected from the group consisting of cellulose-based polymeric materials, carbohydrate-based materials, and materials derived therefrom.

27. The exoprotein inhibitor as set forth in claim 17 further comprising a pharmaceutically active material selected from the group consisting of selective antibacterials, antioxidants, anti-parasitic agents, antipruritics, astringents, local anaesthetics, and anti-inflammatory agents.

28. The exoprotein inhibitor as set forth in claim 17 wherein the non-absorbent substrate is selected from the group consisting of a non-absorbent incontinence device, a barrier birth control device, a non-absorbent contraceptive device, a tampon applicator, and a douche.

29. A tampon applicator for inhibiting the production of exoprotein from Gram positive bacteria comprising a non-absorbent substrate suitable for insertion into the vagina, the non-absorbent substrate having deposited thereon an effective amount of a precursor compound having the general formula: 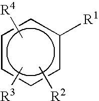
wherein R¹ is selected from the group consisting of 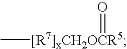
R⁷ is —OCH₂—; X is 0 or 1; R⁵ is a substituted or unsubstituted aromatic ring or a monovalent saturated or unsaturated, substituted or unsubstituted, branched or straight chain hydrocarbyl moiety that may or may not be substituted with hetero atoms; R², R³, and R⁴ are independently selected from the group consisting of H, OH, COOH, wherein upon hydrolysis the precursor compound is capable of producing an active species effective in inhibiting the production of exoprotein from Gram positive bacteria.

30. The tampon applicator as set forth in claim 29 wherein R⁵ is a monovalent saturated, substituted or unsubstituted, branched or straight chain hydrocarbyl moiety that may or may not be substituted with hetero atoms having from 1 to 15 carbon atoms.

31. The tampon applicator as set forth in claim 29 wherein R⁵ is a monovalent saturated, substituted or unsubstituted, branched or straight chain hydrocarbyl moiety that may or may not be substituted with hetero atoms having from 1 to 12 carbon atoms.

32. The tampon applicator as set forth in claim 29 wherein R² is selected from the group consisting of H and OH, and R³ and R⁴ are independently H.

33. The tampon applicator as set forth in claim 29 further comprising a surface-active agent selected from the group consisting of myreth-3-myristate, glycerol monolaurate, and laureth-4.

34. The tampon applicator as set forth in claim 29 wherein the precursor compound is selected from the group consisting of benzyl(s)-(−)-lactate, benzyl ethyl malonate, benzyl-laurate, benzyl benzoate, benzyl paraben, and benzyl salicylate.

35. The tampon applicator as set forth in claim 34 wherein the precursor compound is benzyl(s)-(−)-lactate.

36. The tampon applicator as set forth in claim 35 further comprising a surface-active agent selected from the group consisting of myreth-3-myristate, glycerol monolaurate, and laureth-4.

37. The tampon applicator as set forth in claim 34 wherein the precursor compound is benzyl ethyl malonate.

38. The tampon applicator as set forth in claim 37 further comprising a surface-active agent selected from the group consisting of myreth-3-myristate, glycerol monolaurate, and laureth-4.

39. The tampon applicator as set forth in claim 29 wherein the precursor compound is present in an amount of from about 0.15% (by weight of the non-absorbent substrate) to about 2.0% (by weight of the non-absorbent substrate).

40. The tampon applicator as set forth in claim 29 wherein the precursor compound is present in an amount of from about 0.17% (by weight of the non-absorbent substrate) to about 1.7% (by weight of the non-absorbent substrate).

41. The tampon applicator as set forth in claim 29 wherein the precursor compound is microencapsulated in a shell material.

42. The tampon applicator as set forth in claim 41 wherein the shell material comprises a material selected from the group consisting of cellulose-based polymeric materials, carbohydrate-based materials, and materials derived therefrom.

43. The tampon applicator as set forth in claim 29 further comprising a pharmaceutically active material selected from the group consisting of selective antibacterials, antioxidants, anti-parasitic agents, antipruritics, astringents, local anaesthetics, and anti-inflammatory agents.

44. A tampon applicator for inhibiting the production of exoprotein from Gram positive bacteria comprising a non-absorbent substrate suitable for insertion into the vagina, the non-absorbent substrate having deposited thereon an effective amount of a precursor compound having the general formula: 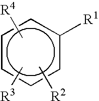
wherein R¹ is 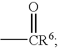
R⁶ is selected from the group consisting of an amino acid, a methyl ester of an amino acid, and an ethyl ester of an amino acid; R², R³, and R⁴ are independently selected from the group consisting of H, OH, COOH, wherein upon hydrolysis the precursor compound is capable of producing an active species effective in inhibiting the production of exoprotein from Gram positive bacteria.

45. The tampon applicator as set forth in claim 44 wherein R⁶ is an amino acid.

46. The tampon applicator as set forth in claim 45 wherein the amino acid is selected from the group consisting of valine, leucine, and cysteine.

47. The tampon applicator as set forth in claim 44 further comprising a surface-active agent selected from the group consisting of myreth-3-myristate, glycerol monolaurate, and laureth-4.

48. The tampon applicator as set forth in claim 44 wherein the precursor compound is selected from the group consisting of n-benzoyl-dl-valine, n-benzoyl-dl-leucine, and n-benzoyl-dl-cysteine.

49. The tampon applicator as set forth in claim 48 further comprising a surface-active agent selected from the group consisting of myreth-3-myristate, glycerol monolaurate, and laureth-4.

50. The tampon applicator as set forth in claim 44 wherein the precursor compound is present in an amount of from about 0.15% (by weight of the non-absorbent substrate) to about 2.0% (by weight of the non-absorbent substrate).

51. The tampon applicator as set forth in claim 44 wherein the precursor compound is present in an amount of from about 0.17% (by weight of the non-absorbent substrate) to about 1.7% (by weight of the non-absorbent substrate).

52. The tampon applicator as set forth in claim 44 wherein the precursor compound is microencapsulated in a shell material.

53. The tampon applicator as set forth in claim 52 wherein the shell material comprises a material selected from the group consisting of cellulose-based polymeric materials, carbohydrate-based materials, and materials derived therefrom.

54. The tampon applicator as set forth in claim 44 further comprising a pharmaceutically active material selected from the group consisting of selective antibacterials, antioxidants, anti-parasitic agents, antipruritics, astringents, local anaesthetics, and anti-inflammatory agents.

55. A douche formulation for inhibiting the production of exoprotein from Gram positive bacteria located in and around a woman's vagina, the douche formulation comprising a vaginal cleansing formulation comprising a pharmaceutically acceptable carrier and an effective amount of a precursor compound having the general formula: 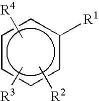
wherein R¹ is selected from the group consisting of 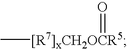
R⁷ is —OCH₂—; X is 0 or 1; R⁵ is a substituted or unsubstituted aromatic ring or a monovalent saturated or unsaturated, substituted or unsubstituted, branched or straight chain hydrocarbyl moiety that may or may not be substituted with hetero atoms; R², R³, and R⁴ are independently selected from the group consisting of H, OH, COOH, wherein upon hydrolysis the precursor compound is capable of producing an active species effective in inhibiting the production of exoprotein from Gram positive bacteria, and wherein the vaginal cleansing formulation is suitable for use in the woman's vagina.

56. The douche formulation as set forth in claim 55 wherein R⁵ is a monovalent saturated, substituted or unsubstituted, branched or straight chain hydrocarbyl moiety that may or may not be substituted with hetero atoms having from 1 to 15 carbon atoms.

57. The douche formulation as set forth in claim 55 wherein R⁵ is a monovalent saturated, substituted or unsubstituted, branched or straight chain hydrocarbyl moiety that may or may not be substituted with hetero atoms from 1 to 12 carbon atoms.

58. The douche formulation as set forth in claim 55 wherein R² is selected from the group consisting of H and OH, and R³ and R⁴ are independently H.

59. The douche formulation as set forth in claim 55 further comprising a surface-active agent selected from the group consisting of myreth-3-myristate, glycerol monolaurate, and laureth-4.

60. The douche formulation as set forth in claim 55 wherein the precursor compound is selected from the group consisting of benzyl(s)-(−)-lactate, benzyl ethyl malonate, benzyl-laurate, benzyl benzoate, benzyl paraben, and benzyl salicylate.

61. The douche formulation as set forth in claim 60 wherein the precursor compound is benzyl(s)-(−)-lactate.

62. The douche formulation as set forth in claim 61 further comprising a surface-active agent selected from the group consisting of myreth-3-myristate, glycerol monolaurate, and laureth-4.

63. The douche formulation as set forth in claim 60 wherein the precursor compound is benzyl ethyl malonate.

64. The douche formulation as set forth in claim 63 further comprising a surface-active agent selected from the group consisting of myreth-3-myristate, glycerol monolaurate, and laureth-4.

65. The douche formulation as set forth in claim 55 wherein the precursor compound is present in the vaginal cleansing formulation in an amount of from about 0.2 millimoles/liter to about 50 millimoles/liter.

66. The douche formulation as set forth in claim 55 wherein the precursor compound is present in the vaginal cleansing formulation in an amount of from about 0.3 millimoles/liter to about 30 millimoles/liter.

67. The douche formulation as set forth in claim 55 wherein the precursor compound is present in the vaginal cleansing formulation in an amount of from about 1.0 millimole/liter to about 15 millimoles/liter.

68. The douche formulation as set forth in claim 55 wherein the precursor compound is microencapsulated in a shell material.

69. The douche formulation as set forth in claim 68 wherein the shell material comprises a material selected from the group consisting of cellulose-based polymeric materials, carbohydrate-based materials, and materials derived therefrom.

70. The douche formulation as set forth in claim 55 further comprising a pharmaceutically active material selected from the group consisting of selective antibacterials, antioxidants, anti-parasitic agents, antipruritics, astringents, local anaesthetics, and anti-inflammatory agents.

71. A douche formulation for inhibiting the production of exoprotein from Gram positive bacteria located in and around a woman's vagina, the douche formulation comprising a vaginal cleansing formulation comprising a pharmaceutically acceptable carrier and an effective amount of a precursor compound having the general formula: 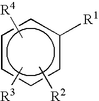
wherein R¹ is 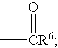
R⁶ is selected from the group consisting of an amino acid, a methyl ester of an amino acid, and an ethyl ester of an amino acid; R², R³, and R⁴ are independently selected from the group consisting of H, OH, COOH, wherein upon hydrolysis the precursor compound is capable of producing an active species effective in inhibiting the production of exoprotein from Gram positive bacteria, and wherein the vaginal cleansing formulation is suitable for use in the woman's vagina.

72. The douche formulation as set forth in claim 71 wherein R⁶ is an amino acid.

73. The douche formulation as set forth in claim 72 wherein the amino acid is selected from the group consisting of valine, leucine, and cysteine.

74. The douche formulation as set forth in claim 71 further comprising a surface-active agent selected from the group consisting of myreth-3-myristate, glycerol monolaurate, and laureth-4.

75. The douche formulation as set forth in claim 71 wherein the precursor compound is selected from the group consisting of n-benzoyl-dl-valine, n-benzoyl-dl-leucine, and n-benzoyl-dl-cysteine.

76. The douche formulation as set forth in claim 75 further comprising a surface-active agent selected from the group consisting of myreth-3-myristate, glycerol monolaurate, and laureth-4.

77. The douche formulation as set forth in claim 71 wherein the precursor compound is present in the vaginal cleansing formulation in an amount of from about 0.2 millimoles/liter to about 50 millimoles/liter.

78. The douche formulation as set forth in claim 71 wherein the precursor compound is present in the vaginal cleansing formulation in an amount of from about 0.3 millimoles/liter to about 30 millimoles/liter.

79. The douche formulation as set forth in claim 71 wherein the precursor compound is present in the vaginal formulation in an amount of from about 1.0 millimole/liter to about 15 millimoles/liter.

80. The douche formulation as set forth in claim 71 wherein the precursor compound is microencapsulated in a shell material.

81. The douche formulation as set forth in claim 80 wherein the shell material comprises a material selected from the group consisting of cellulose-based polymeric materials, carbohydrate-based materials, and materials derived therefrom.

82. The douche formulation as set forth in claim 71 further comprising a pharmaceutically active material selected from the group consisting of selective antibacterials, antioxidants, anti-parasitic agents, antipruritics, astringents, local anaesthetics, and anti-inflammatory agents.
